(19) United States
(12) Patent Application Publication
(10) Pub. No.: US 2020/0294651 A1
(43) Pub. Date: Sep. 17, 2020

(54) SLEEP IMPROVEMENT ASSISTANCE SYSTEM, METHOD, AND PROGRAM

(71) Applicant: NEC Solution Innovators, Ltd., Koto-ku, Tokyo (JP)

(72) Inventor: Jou AKITOMI, Koto-ku, Tokyo (JP)

(73) Assignee: NEC Solution Innovators, Ltd., Koto-ku, Tokyo (JP)

(21) Appl. No.: 16/753,576

(22) PCT Filed: Sep. 5, 2018
(86) PCT No.: PCT/JP2018/032851

§ 371 Date: Apr. 3, 2020

(30) Foreign Application Priority Data
Oct. 17, 2017 (JP) 2017-200933

(51) Int. Cl. G16H 20/70; G16H 10/60; G16H 50/70; G16H 70/20; G16H 50/20; A61B 5/00; A61B 5/16

(52) U.S. Cl. CPC G16H 20/70; G16H 10/60; G16H 50/70; A61B 5/165; G16H 50/20; A61B 5/4848; A61B 5/4815; G16H 70/20

(57) ABSTRACT

A sleep improvement assistance system 600 of the present invention includes: an information providing unit 601 that uses an automatic discrimination model that automatically determines and outputs, when user information that is information regarding sleep of a target user is inputted, an output suitable for the target user from a predetermined output set in accordance with a phase of a sleep improvement program of the target user, to provide information to the target user; a result data storage unit 602 that stores result data including at least user information and information regarding executed information provision, for a past user who has finished a sleep improvement program; and a criterion correction unit 603 that compares, in using an automatic determination model, user information of the target user with user information included in the result data, and corrects a determination criterion for an output of the automatic discrimination model.

### TECHNICAL FIELD

The present invention relates to a sleep improvement assistance system, a sleep improvement assistance method, and a sleep improvement assistance program that assist a user's sleep improvement activity.

### BACKGROUND ART

Many cognitive behavioral therapies for insomnia (CBT-I) are conducted at clinical sites through counseling of experts such as doctors and therapists, based on records in sleep diaries. CBT-I is a globally used technique that is also effective in reducing the use of sleeping pills, but there is currently a shortage of experts who can provide the technique in Japan.

Here, cognitive behavioral therapy (CBT) is a psychotherapy aimed at making cognitive and behavioral habits controllable by reviewing, and is conducted through patient's own task execution under education of doctors and therapists. While the number of potential patients with chronic insomnia disorder (psychophysiological insomnia) is said to be about 3 million, there is also data that the effect of CBT-I has been observed in about 70% of patients for which application of CBT-I is determined to be effective, and there is a demand for widespread use as a therapy with a high remission rate, a long-lasting effect, and a dose reduction effect.

In recent years, various researches and developments for adopting IT tools have been performed to enable CBT-I to be provided to many people.

FIG. 22 is an explanatory diagram showing an example of a process of CBT-I. In CBT-I, for example, as shown in FIG. 22, after education from a doctor, task setting, task execution, recording in a sleep diary, and feedback are repeatedly performed during a predetermined period. At that time, by checking the effect and adding or resetting the task as appropriate through the feedback, cognitive and behavioral habits leading to insomnia are improved, and the sleep state is improved.

As an example of a method for adopting IT tools for the process of the CBT-I, there is a method of performing all processes non-face-to-face, such as the Web and e-mail, by accumulating expert's know-how as data and selecting and providing data that meets conditions from the accumulated data.

Regarding a technique for adopting IT tools for such activities by experts, for example, PTL 1 describes an example of a health management server that provides, with use of IT, a health guidance service that has been given in face-to-face with an expert.

### CITATION LIST

### Patent Literature

PTL 1: Japanese Patent No. 6010719

### SUMMARY OF INVENTION

### Technical Problem

However, when trying to implement 100% IT tooling that do not require any expert intervention, the following problems occur.

First, in order to automatically select an optimal task for each user from among many tasks, a clear determination criterion for determining how suitable each task is for the user is required. However, it is difficult to appropriately set such a clear determination criterion.

Clinical task selection is made on the basis of experiences of experts and there is no clear determination criterion. In order to reproduce such empirical selection with a machine, there is a problem such as requiring a huge number of samples. Further, even if the huge number of samples can be prepared, determination using a statistically calculated determination criterion is not always optimal for each user.

Second, there is a problem of how to consider psychological effects. In CBT-I, in addition to the selection of tasks, there are advices and the like that are given on the basis of knowledge of experts, in order to assist patients from a psychological aspect. An example is message transmission such as praising a patient for improving motivation to continue or pointing out cognitive or behavioral problems. It is particularly difficult to appropriately set a clear determination criterion for such advices and the like relating to psychological effects on the user.

Note that the method described in PTL 1 obtains a degree of confidence indicating certainty of answer information extracted from message information transmitted from a terminal, and provides evaluation based on the degree of confidence to a user after correction based on information of the user. According to the method described in PTL 1, from a tendency value of a past behavior of the user, for example, the evaluation of each task can be changed for each user by using a correction value obtained by giving a negative weight to an index of “load” of the task.

However, as described in PTL 1, the method of changing evaluation of a task on the basis of a tendency value of a past behavior of a user has a problem of requiring information regarding the past behavior of the user, and being unable to be applied to a task to be presented first. Meanwhile, PTL 1 describes that a correlation coefficient between a characteristic value of a user's living body and a value of any index can be obtained, and correction can be performed using the correlation coefficient. However, information used for obtaining such a correlation coefficient is information of a past user, and the obtained correlation coefficient does not always match the user.

In view of the problems described above, it is an object of the present invention to provide a sleep improvement assistance system, a sleep improvement assistance method, and a sleep improvement assistance program that can optimize and provide, for each user, various processes that have been performed by experts in sleep improvement activities.

### Solution to Problem

A sleep improvement assistance system according to the present invention includes: an information providing unit that uses an automatic discrimination model that automatically determines and outputs, when user information that is information regarding sleep of a target user of a sleep improvement program based on CBT-I is inputted, an output suitable for the target user from a predetermined output set in accordance with a phase of a sleep improvement program of the target user, to provide information to the target user; a result data storage unit that stores result data including at least user information and information regarding information provision performed by the information providing unit, for a past user who has finished a sleep improvement program; and a criterion correction unit that compares user information of the target user with user information included in the result data, and corrects a criterion to be used when the automatic discrimination model determines an output suitable for a user, on the basis of a result of the comparison. The information providing unit provides information to the target user by using the automatic discrimination model after the criterion is corrected by the criterion correction unit.

Further, a sleep improvement assistance method according to the present invention includes, by an information processing device: using an automatic discrimination model that automatically determines and outputs, when user information that is information regarding sleep of a target user of a sleep improvement program based on CBT-I is inputted, an output suitable for the target user from a predetermined output set in accordance with a phase of a sleep improvement program of the target user, to provide information to the target user; storing, in a predetermined result data storage unit, result data including at least user information and information regarding information provision performed by the information processing device in a sleep improvement program, for a past user who has finished a sleep improvement program; and comparing, in using the automatic discrimination model, user information of the target user with user information included in the result data, and correcting a criterion to be used when the automatic discrimination model determines an output suitable for a user, on the basis of a result of the comparison.

In addition, a sleep improvement assistance program according to the present invention causes a computer to execute: a process of using an automatic discrimination model that automatically determines and outputs, when user information that is information regarding sleep of a target user of a sleep improvement program based on CBT-I is inputted, an output suitable for the target user from a predetermined output set in accordance with a phase of a sleep improvement program of the target user, to provide information to the target user; a process of storing, in a predetermined result data storage unit, result data including at least user information and information regarding information provision performed by the computer in a sleep improvement program, for a past user who has finished a sleep improvement program; and a process of comparing, in using the automatic discrimination model, user information of the target user with user information included in the result data, and correcting a criterion to be used when the automatic discrimination model determines an output suitable for a user, on the basis of a result of the comparison.

### Advantageous Effects of Invention

According to the present invention, various processes that have been performed by experts in sleep improvement activities can be optimized and provided for each user.

### BRIEF DESCRIPTION OF DRAWINGS

FIG. 1 It depicts a schematic configuration diagram of a sleep improvement assistance system according to a first exemplary embodiment.

FIG. 2 It depicts a flowchart showing an operation example of the sleep improvement assistance system of the first exemplary embodiment.

FIG. 3 It depicts a block diagram showing a configuration example of a sleep improvement assistance system according to a second exemplary embodiment.

FIG. 4 It depicts a block diagram showing a configuration example of a task setting unit 27.

FIG. 5 It depicts a block diagram showing a configuration example of a notification unit 28.

FIG. 6 It depicts a block diagram showing a configuration example of a feedback unit 29.

FIG. 7 It depicts a flowchart showing an example of an operation of the sleep improvement assistance system according to the second exemplary embodiment.

FIG. 8 It depicts a flowchart showing an example of a more detailed processing flow of a task selection process.

FIG. 9 It depicts a flowchart showing an example of a more detailed processing flow of a notification determination process.

FIG. 10 It depicts a flowchart showing an example of a more detailed processing flow of a feedback determination process.

FIG. 11 It depicts an explanatory view showing an example of information stored in a personal DB 24.

FIG. 12 It depicts an explanatory view showing an example of information stored in a result DB 26.

FIG. 13 It depicts an explanatory view showing an example of question items related to a user's lifestyle and sleep state.

FIG. 14 It depicts an explanatory view showing an example of a sleep improvement action corresponding to each item of the question items.

FIG. 15 It depicts an explanatory view showing an example of information stored in a task DB 21.

FIG. 16 It depicts an explanatory view showing an example of searching for a similar user.

FIG. 17 It depicts an explanatory view showing an example of calculating an individual effectiveness of a similar user.

FIG. 18 It depicts an explanatory view showing an example of presenting a task to a target user.

FIG. 19 It depicts an explanatory view showing an example of presenting a task to a target user.

FIG. 20 It depicts a schematic block diagram showing a configuration example of a computer according to each exemplary embodiment of the present invention.

FIG. 21 It depicts a block diagram showing an outline of a sleep improvement assistance system of the present invention.

FIG. 22 It depicts an explanatory diagram showing an example of a process of CBT-I.

### DESCRIPTION OF EMBODIMENTS

### First Exemplary Embodiment

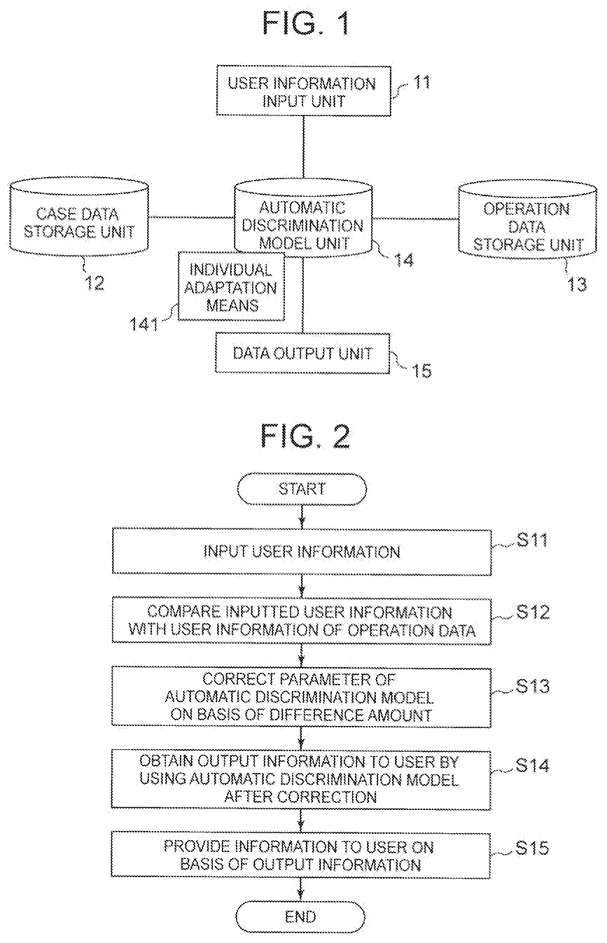

Hereinafter, an exemplary embodiment of the present invention will be described with reference to the drawings. FIG. 1 is a schematic configuration diagram of a sleep improvement assistance system according to a first exemplary embodiment. As shown in FIG. 1, the sleep improvement assistance system of the present exemplary embodiment is a system that provides a sleep improvement program that is a program in which a process of CBT-I is performed only by a user without intervention of an expert. The sleep improvement assistance system is roughly sectioned into five functional units, namely, a user information input unit 11, a case data storage unit 12, an operation data storage unit 13, an automatic discrimination model unit 14, and a data output unit 15.

The user information input unit 11 inputs information regarding sleep of a user as a target of sleep improvement (hereinafter, simply referred to as user information), such as information regarding a lifestyle of the user.

The case data storage unit 12 stores case data such as an example of outputs (information provision) performed by experts to individuals. Here, the outputs performed by experts include any information provision based on knowledge of experts, such as, for example, presenting a task for a lifestyle of an individual, and presenting a comment (advice, encouragement, commentary, and the like) for a task execution status, a comment after the task execution, and presentation of a next task.

The case data storage unit 12 may associate and store personal information corresponding to an input of an automatic discrimination model, which will be described later, and output information of the expert corresponding to an output of the automatic discrimination model.

For example, when the automatic discrimination model is a model that outputs a task suitable for a user in response to an input of information regarding the user's lifestyle, the case data storage unit 12 may store a task presented by an expert to an individual as case data in association with information regarding a lifestyle of an individual.

In addition, for example, when the automatic discrimination model is a model that outputs a comment suitable for a user in response to an input of information regarding a task execution status, the case data storage unit 12 may store a comment made by an expert on an individual as case data in association with information regarding a task execution status of the individual.

In addition, for example, when the automatic discrimination model is a model that outputs a comment or a next task suitable for a user in response to an input of information regarding a status after task execution, the case data storage unit 12 may store a comment made by an expert or a next task presented to an individual as case data in association with information regarding a status of the individual after the task execution.

The operation data storage unit 13 stores, as actual data (also referred to as operation data), information (hereinafter, referred to as output information) regarding an output actually performed by the system for the user and information obtained from the user. The operation data storage unit 13 may store output information in association with, for example, user information inputted in the past. Note that the user information may include information obtained from the user in each phase of a sleep improvement program, for example, information regarding a set task, a task execution status, information regarding a sleep improvement status, and the like. Further, the output information may include, for example, output contents (information provided by the system) obtained by the automatic discrimination model to be described later, output timing, a criterion when these are selected, and the like.

The automatic discrimination model unit 14 holds an automatic discrimination model obtained by learning outputs of experts, and determines, when user information of a certain user is inputted, an output suitable for the user by using the held automatic discrimination model. The automatic discrimination model is constructed on the basis of case data stored in the case data storage unit 12, for example.

As shown in FIG. 1, the automatic discrimination model unit 14 of the present exemplary embodiment includes an individual adaptation means 141. When using the automatic discrimination model, the individual adaptation means 141 optimizes (individually adapts) a parameter of the automatic discrimination model on the basis of the inputted user information and operation data stored in the operation data storage unit 13. This allows the automatic discrimination model to determine an output (more specifically, output contents, output timing, and the like) suitable for the user.

When user information is inputted, the individual adaptation means 141 corrects a parameter of the automatic discrimination model to be optimized for the user, on the basis of the inputted user information and the operation data stored in the operation data storage unit 13. For example, on the basis of a difference amount (or a similarity degree that is a degree of similarity) obtained by comparing the user information and user information of a past user included in the operation data, the individual adaptation means 141 corrects the automatic discrimination model. At this time, the individual adaptation means 141 may select past user information to be used for correction, or adjust a parameter correction amount in accordance with the obtained difference amount or similarity degree.

Here, the automatic discrimination model may be a model that, when user information is inputted, at least selects and outputs output contents suitable for the user from a predetermined set. In such a case, for the user of the inputted user information, the individual adaptation means 141 optimizes a criterion (hereinafter, referred to as a selection criterion) for selecting output contents that the automatic discrimination model has as a parameter.

Further, for example, the automatic discrimination model may be a model that, when user information is inputted, selects and outputs output contents when a predetermined output condition of output contents is satisfied. In such a case, for the user of the inputted user information, the individual adaptation means 141 optimizes a criterion (hereinafter, referred to as an execution criterion) for selecting output contents and output timing that the automatic discrimination model has as a parameter.

The output contents selected by the automatic discrimination model are: for example, a candidate for a task to be worked on by the user in the sleep improvement program provided by this system; a comment on a task execution status; a comment on a status after the task execution; or a candidate for a next task.

Further, the automatic discrimination model unit 14 appropriately updates the automatic discrimination model by using the operation data stored in the operation data storage unit 13.

The data output unit 15 provides information to the user on the basis of contents outputted from the automatic discrimination model unit 14 (output contents obtained by the automatic discrimination model).

Next, an operation of the present exemplary embodiment will be described. FIG. 2 is a flowchart showing an operation example of the sleep improvement assistance system of the present exemplary embodiment. Note that, in the example shown in FIG. 2, it is assumed that a new user starts using this system in a state where the automatic discrimination model (initial model) has already been constructed on the basis of case data, and then learning of the automatic discrimination model is appropriately performed with use of the operation data.

Note that the operation data storage unit 13 stores, as operation data, at least user information inputted to the automatic discrimination model, output information, and information regarding effects (for example, information regarding a sleep improvement status after execution of the program) as actual data, for users who have used this system so far. Further, it is assumed that the output information includes, in addition to output contents obtained by the automatic discrimination model, information on parameters used at that time.

In this example, first, the user information input unit 11 inputs user information (step S11).

Next, the individual adaptation means 141 compares the inputted user information with user information of the operation data, and calculates a difference amount (step S12). As a method of comparing the user information, there are a method of individually comparing each item of the inputted user information with each item of the user information of the operation data to determine a difference between them and calculating a sum of the differences, and a method of calculating a feature vector from the user information and obtaining a distance between the feature vectors.

At this time, a target to be compared with the inputted user information may be all the user information of the operation data or a part of the user information. For example, the individual adaptation means 141 may compare only user information having a certain similarity degree or more with the input user information among the user information of the operation data.

Next, the individual adaptation means 141 corrects a parameter of the automatic discrimination model on the basis of the obtained difference amount (step S13). The parameter of the automatic discrimination model to be corrected is not particularly limited. The parameter may be a set value (fixed value) that has been set on the basis of knowledge of experts, and may be a variable and the like obtained by machine learning and the like.

In step S13, the individual adaptation means 141 can also correct the parameter of the automatic discrimination model on the basis of the obtained difference amount and information regarding the effect on the user as the comparison object.

For example, when the parameter of the automatic discrimination model includes a selection criterion, the individual adaptation means 141 may correct the selection criterion. Further, for example, when the parameter of the automatic discrimination model includes an execution criterion, the individual adaptation means 141 may correct the execution criterion. At this time, when the difference amount is obtained for each item, it is also possible to correct a value corresponding to the item in the parameter on the basis of a difference amount of the item and information (improvement degree, and the like) regarding the effect on the user as the comparison object at that time.

Note that the above is an example of a case where a parameter such as a selection criterion is explicitly included. However, for example, the following correction is also possible. That is, when the automatic discrimination model outputs, as a parameter, a transition probability between states or a function at a time of the state transition, it is possible to correct a coefficient, weight, and the like in a calculation formula used in calculating the transition probability and the function, on the basis of a difference amount and an effect on the user for whom the difference amount has been obtained. Regardless of whether it is thus explicit or not, an operation of correcting a value of an index to be used for a determination criterion as a result is also included in correction of the selection criterion or the execution criterion in a broad sense.

Note that the “difference amount” described above may be read as “similarity degree”. In this case, the similarity degree may be simply evaluated to be higher as the difference amount is smaller.

Next, the automatic discrimination model unit 14 inputs the inputted user information to the automatic discrimination model after correction, and obtains output information (output contents and output timing) to the user (step S14).

Finally, the data output unit 15 provides information to the user on the basis of the output information obtained by the automatic discrimination model unit 14 (step S15).

As described above, in the present exemplary embodiment, in using the automatic discrimination model that has learned outputs of experts, the automatic discrimination model is used after parameters are individually adapted to individual users. Therefore, information for sleep improvement can be provided to the user. An optimal sleeping habit often differs from user to user. For this reason, in the present exemplary embodiment, the parameter of the automatic discrimination model is optimized for each user on the basis at least of a difference amount between information on the user and information on another user.

Therefore, it is possible to provide information for sleep improvement more suitable for individual users without manual intervention. As a result, effects similar to those obtained by experts are expected, such as, for example, improvement of a user's sleep state, and enhancement of motivation to continue the sleep improvement program and motivation to improve the lifestyle after the end of the sleep improvement program.

### Second Exemplary Embodiment

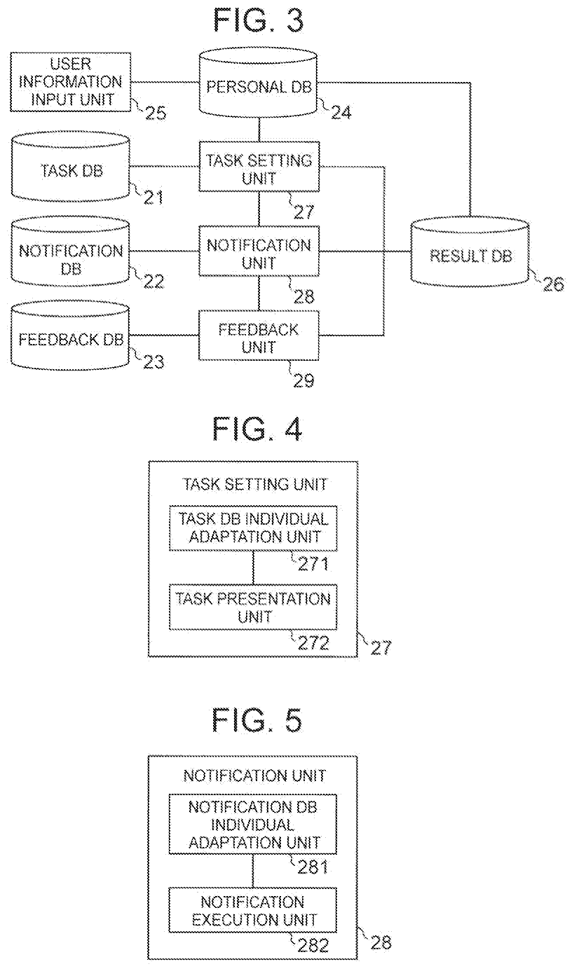

Next, a second exemplary embodiment of the present invention will be described. FIG. 3 is a block diagram showing a configuration example of a sleep improvement assistance system according to the second exemplary embodiment. The sleep improvement assistance system shown in FIG. 3 includes a task database (DB) 21, a notification DB 22, a feedback DB 23, a personal DB 24, a user information input unit 25, a result DB 26, a task setting unit 27, and a notification unit 28, and a feedback unit 29.

The task DB 21 stores information regarding a task for sleep improvement presented by this system to the user. The task DB 21 stores, for example, a standard selection criterion (an effectiveness, an execution difficulty, and the like) for user information for each task. The term “standard” as used herein means being statistically processed in a broad sense based on knowledge of experts, machine learning, and the like, that is, means that no specific individual circumstance of each user is considered.

In CBT-I, an effectiveness of a task is determined in accordance with a lifestyle of the individual. Therefore, based on knowledge of experts, for a group of items of information regarding a lifestyle collected from a user, a standard effectiveness of each task may be determined for each value range of each item, and stored in the task DB 21. Note that the standard effectiveness of the task is used as a priority in presenting to the user.

Further, on the basis of the knowledge of experts, a standard execution difficulty of each task is determined in advance and stored in the task DB 21.

The notification DB 22 stores information regarding notification performed by this system to a user during a task execution period or the like. Here, the notification is, for example, an output of a message having a content that enhances motivation to continue the sleep improvement program, such as encouraging task execution or praising an improvement state of insomnia and the like. Examples of an output method include outputting to a screen, mail transmission, and the like.

The notification DB 22 stores, for example, a standard determination criterion for a notification content and notification timing for a task execution status and a sleep improvement status during a task execution period. The notification DB 22 may store, for example, a standard determination criterion for a notification content and notification timing for a task execution status and a sleep improvement status for each task. Further, for example, the notification DB 22 may store, for each notification content, a standard determination criterion (execution criterion) for a task execution status and a sleep improvement status.

The standard determination criterion for a notification content is a criterion for determining whether to perform notification with the notification content, and may be any information as long as it is for determining whether to output a specific notification content from a task execution status or a sleep improvement status during task execution. The criterion may be, for example, a condition (a threshold value, conditional expression, or the like) for the task execution status or the sleep improvement status during execution of the task, for selecting one or more notification contents from a predetermined set of notification contents for the task being executed.

Examples of such a criterion include, for example, a criterion for encouraging task execution, and a criterion for praising an improvement status of insomnia or the like

Further, the standard determination criterion for notification timing is a criterion for determining when to notify a certain notification content, and may be any information as long as it is for determining output timing of a specific notification content from an execution status or a sleep improvement status. The criterion may be an effectiveness or a condition (a threshold value, conditional expression, or the like) for the task execution status or the sleep improvement status during execution of the task, for determining output timing of a specific notification content predetermined for the task being executed. Note that, regardless of the task being executed, this criterion may be a condition for the task execution status or the sleep improvement status during execution of the task, for selecting a content from a predetermined set of specific notification contents with notification timing.

Examples of such a criterion include: a threshold value of a duration that defines the number of days of interruption of task execution or diary recording before giving notification; a threshold value of an improvement degree that defines a degree of improvement of a sleep status before giving notification; and the like.

Note that the determination criterion for the notification content and the determination criterion for the notification timing need not be clearly distinguished. That is, it is also possible to determine that the determination criterion for the notification timing is satisfied when a determination criterion for a certain notification content is satisfied.

Such determination criteria for the notification content and the notification timing allow specific determination as to what message is to be notified at what timing, for a specific execution status and improvement status.

The feedback DB 23 stores information regarding feedback performed by this system to a user after the end of a task execution period or the like. Here, the feedback is, for example, presentation of a task or output of a message with a content for continuing motivation to improve a lifestyle after the end of the sleep improvement program, such as praising or pointing out the task, for a status at the end of the task.

The feedback DB 23 stores, for example, a standard determination criterion for feedback contents (items for praising, items for pointing out as a task, and the like) for a task execution status and a sleep improvement status at the end of the task execution period. The feedback DB 23 may store, for example, for each task, a standard determination criterion for a feedback content, for a task execution status and a sleep improvement status after the task execution. Further, for example, the feedback DB 23 may store, for each feedback content, a standard determination criterion (selection criterion) for the task execution status and the sleep improvement status.

The standard determination criterion for a feedback content is a criterion for determining whether to perform feedback with the feedback content, and may be any information as long as it is for determining whether to output a specific feedback content from a task execution status or a sleep improvement status after the task execution. The criterion may be, for example, an effectiveness or a condition (a threshold value, conditional expression, or the like) for the task execution status or the sleep improvement status after execution of the task, for selecting one or more feedback contents from a predetermined set of feedback contents for a set task.

Examples of such a criterion include a determination criterion for items for praising and items for pointing out as a task, and more specifically, a threshold value and the like for determining quality of the sleep improvement status, and a threshold value and the like for determining quality of the task execution status.

Based on such a determination criterion for the feedback content, it is specifically determined what message is to be outputted as feedback for a specific execution status or improvement status.

The personal DB 24 stores user's personal data. The user's personal data includes data related to user's personal sleep. In the present exemplary embodiment, the user's personal data is referred to as user information. The user information may include, for example, in addition to personal attributes such as gender and age, (a) daily sleep record, (b) a sleep-related lifestyle, (c) a degree of insomnia, (d) a sleep-related task, (e) a daytime activity status, (f) a desired self-image, and the like.

Examples of (a) daily sleep record include the following.
Time of entering the bed
Time of falling asleep
Time of getting out of the bed
The number of times of waking up in a middle
A total time of being awake in a middle
A total time of a daytime nap
A difference of sleeping time between a weekday and a weekend
A difference of time of waking up
A difference of time of falling asleep
A difference of a central time during sleep

In addition, examples of (b) a sleep-related lifestyle include the following.
Information regarding actions from waking up to getting up in the morning Specific example: whether having opened a curtain when getting up in the morning
Information regarding actions for sunbathing
Information regarding daytime sleep, temporary sleep, and the like
Information regarding how to spend a weekend
Information regarding a habit of taking caffeinated beverages

Further, examples of (c) a degree of insomnia include Athens Insomnia Scale (AIS), Insomnia Severity Index (ISI), and the like that are internationally used and calculated from questionnaires and the like.

Further, examples of (d) a sleep-related task include a task that is being selected, an achievement degree of the selected task for each day, and the like. Note that the number of the selected tasks is not limited to one, but may be plural.

In addition, (e) a daytime activity status may simply be an index that shows a degree of being active.

Further, (f) a desired self-image is used as a slogan or used for classifying user attributes and the like.

The user information input unit 25 appropriately inputs personal data (user information) of a user to be assisted by this system, and updates the personal DB 24. Hereinafter, the user to be assisted by this system may be referred to as a target user.

The result DB 26 stores result data indicating a result of a user who has finished a sleep improvement program. The result data according to the present exemplary embodiment includes, for example, information regarding a task presented by the system, notification performed by the system, feedback performed by the system, and the like, in addition to the user's personal data. Further, the user's personal data includes information regarding the user in each phase of the sleep improvement program, for example, information regarding a lifestyle and a sleep status before setting the task, a lifestyle and a sleep status during each task execution period, the selected task and an execution status thereof, and an improvement status after the end of the task.

Here, examples of the improvement status after the end of the task include an improvement status of a degree of insomnia based on a questionnaire, a sleep improvement status based on a sleeping record, a sleep improvement status based on daytime activity status, and the like. Further, the information regarding the task presented by the system may include not only the presented task but also a task selection criterion. In addition, the information regarding notification performed by the system may include not only a notification content and notification timing but also a determination criterion for the notification content and the notification timing. Furthermore, the information regarding feedback performed by the system may include not only a feedback content but also a determination criterion for the feedback content.

The task setting unit 27 acquires the user information stored in the personal DB 24, selects and presents a task that is effective for the user from among the tasks stored in the task DB 21, and sets the task to be executed by the user.

FIG. 4 is a block diagram showing a configuration example of the task setting unit 27. As shown in FIG. 4, the task setting unit 27 may include a task DB individual adaptation unit 271 and a task presentation unit 272.

As optimization processing for the target user, the task DB individual adaptation unit 271 corrects a task selection criterion (specifically, a standard effectiveness, a standard execution difficulty, and the like that are used as indices for the task selection criterion) stored in the task DB 21, on the basis of the user information stored in the personal DB 24 and the result data stored in the result DB 26.

The task presentation unit 272 uses the selection criterion corrected by the task DB individual adaptation unit 271, to select, from the tasks stored in the task DB 21, and present an effective task for the user information stored in the personal DB 24. In addition, the task presentation unit 272 finally sets a task to be executed by the user, for example, by accepting a user input for the presented task.

The notification unit 28 acquires the user information stored in the personal DB 24, and performs notification effective for the user on the basis of the information stored in the notification DB 22.

FIG. 5 is a block diagram showing a configuration example of the notification unit 28. As shown in FIG. 5, the notification unit 28 may include a notification DB individual adaptation unit 281 and a notification execution unit 282.

As optimization processing for the target user, the notification DB individual adaptation unit 281 corrects a determination criterion for a notification content and notification timing stored in the notification DB 22, on the basis of the user information stored in the personal DB 24 and the result data stored in the result DB 26.

The notification execution unit 282 uses the determination criterion corrected by the notification DB individual adaptation unit 281 to determine an effective notification content for the user from the notification contents stored in the notification DB 22 and the notification timing thereof, on the basis of the user information stored in the personal DB 24, and actually performs the notification.

The feedback unit 29 acquires the user information stored in the personal DB 24, and performs effective feedback for the user on the basis of the information stored in the feedback DB 23.

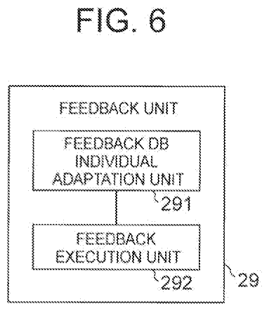

FIG. 6 is a block diagram showing a configuration example of the feedback unit 29. As shown in FIG. 6, the feedback unit 29 may include a feedback DB individual adaptation unit 291 and a feedback execution unit 292.

As optimization processing for the target user, the feedback DB individual adaptation unit 291 corrects a determination criterion for a feedback content stored in the feedback DB 23, on the basis of the user information stored in the personal DB 24 and the result data stored in the result DB 26.

The feedback execution unit 292 uses the determination criterion corrected by the feedback DB individual adaptation unit 291 to select an effective feedback content for the user from among the feedback contents stored in the feedback DB 23, on the basis of the user information stored in the personal DB 24, and actually performs the feedback.

Note that, in the present exemplary embodiment, each of the task presentation unit 272, the notification execution unit 282, and the feedback execution unit 292 corresponds to the automatic discrimination model of the first exemplary embodiment, and the above-mentioned criterion (for example, a task selection criterion, a determination criterion for a notification content and notification timing, and a determination criterion for a feedback content) used by these for determining an output content and timing thereof corresponds to a parameter of the automatic discrimination model.

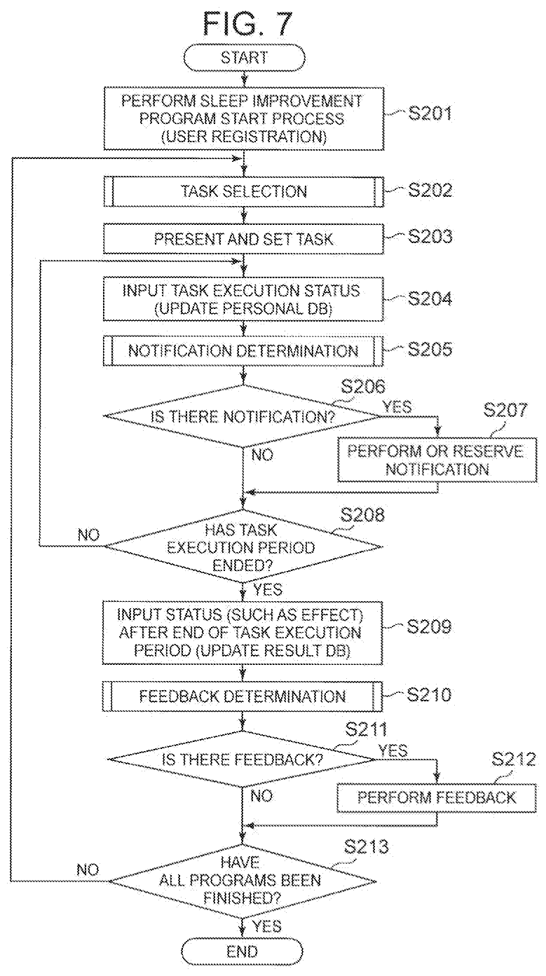

Next, an operation of the present exemplary embodiment will be described. FIG. 7 is a flowchart showing an example of an operation of the sleep improvement assistance system of the present exemplary embodiment. The operation shown in FIG. 7 is an example of an operation from when a user joins this system until the end of the sleep improvement program. In this example, it is assumed that the task DB 21, the notification DB 22, and the feedback DB 23 store information as a standard determination criterion obtained through knowledge of experts or machine learning in advance, regarding the task, the notification, and the feedback.

First, the user information input unit 25 performs a sleep improvement program start process in response to a request from a user (step S201). The user information input unit 25 uses, for example, a user information input screen and the like for starting a program, to acquire and register user's personal data (user information) in the personal DB 24. At this time, the user information input unit 25 may assign a user ID for identifying an individual to the user, and register the personal data in association with the assigned user ID.

When the start process is completed, the task setting unit 27 performs a task selection process (step S202). In this process, as will be described in detail later, regarding a task, the task is selected on the basis of a criterion optimized for the target user.

Next, the task presentation unit 272 of the task setting unit 27 presents the selected task to the user, and sets the task to be executed by the user in the sleep improvement program provided by this system (step S203). The task presentation unit 272 may simply set the task by, for example, along with the presentation of the task, inquiring about right or wrong of the task, receiving an input for the inquiry, and the like. Further, when the task is set, the task presentation unit 272 may update user information stored in the personal DB 24, and temporarily register the user information of the target user in the result DB 26 as result data.

When the setting of the task is completed, the sleep improvement program shifts to a task execution phase by the user.

In the task execution phase, the user inputs a task execution status every day (step S204). In step S204, the user information input unit 25 uses, for example, a user information input screen and the like for the execution phase, to acquire and register a task execution status of the user as a part of the user information in the personal DB 24.

Next, at predetermined timing, the notification unit 28 determines notification (step S205). Here, examples of the predetermined timing include a fixed cycle such as each day and each time the execution status is inputted. In this process, as will be described in detail later, regarding a notification, the presence or absence of notification is determined on the basis of a criterion optimized for the target user, and a notification content and notification timing thereof are determined when there is notification.

Next, when it is determined that there is notification as a result of the determination (Yes in step S206), the notification execution unit 282 of the notification unit 28 executes or reserves the notification in accordance with the determined notification content and notification timing (step S207). Here, the reservation of the notification is to reserve message transmission or mail transmission such that a message or a mail of the notification content is transmitted at the specified timing. In addition, when executing or reserving the notification, the notification execution unit 282 temporarily registers information on the performed notification (including information of the used criterion) in the result DB 26 as result data of the user, along with a user's task execution status (continuation status) obtained so far. Thereafter, the process proceeds to step S208.

Whereas, when it is determined that there is no notification (No in step S206), the process directly proceeds to step S208.

In step S208, it is determined whether or not the task execution period has ended. When the task execution period has not ended (No in step S208), the process returns to step S204 and waits until a next execution status is inputted. Whereas, when the task execution period has ended (Yes in step S208), the user information input unit 25 temporarily registers, in the result DB 26 as result data of the user, a user's task execution status (achievement status) and improvement status after execution obtained so far. Thereafter, the process proceeds to step S209. Note that the sleep improvement program shifts to an evaluation phase when the task execution period ends.

In the evaluation phase, the user inputs a status after the end of the task execution period (step S209). In step S209, the user information input unit 25 uses, for example, a user information input screen and the like for the evaluation phase, to acquire the status after the end of the task execution period of the user as a part of the user information, and register in the personal DB 24.

Next, the feedback unit 29 determines feedback (step S210). In the process, as will be described in detail later, regarding the feedback, the presence or absence of feedback is determined and a content of the feedback is determined when there is the feedback, on the basis of a criterion optimized for the user.

Next, when it is determined that there is feedback as a result of the determination (Yes in step S211), the feedback execution unit 292 of the feedback unit 29 performs the feedback in accordance with the determined feedback content (step S212). Further, when executing the feedback, the feedback execution unit 292 temporarily registers information on the performed feedback (including information on the used criterion) in the result DB 26 as result data of the user, along with a status (improvement status, and the like) after the user's task execution obtained so far. Thereafter, the process proceeds to step S213.

Whereas, when it is determined that there is no notification (No in step S211), the process directly proceeds to step S213.

In step S213, it is determined whether or not all of the sleep improvement programs for the user have been finished. When not all of the sleep improvement programs have been finished (No in step S213), the process returns to step S202 to perform a next task selection process. Whereas, when all of the sleep improvement programs have been finished (Yes in step S213), processing for the user is finished.

Note that the information regarding the target user temporarily registered in the result DB 26 may be permanently registered as result data when the sleep improvement program is finished. The timing and the like for registering the result data in the result DB 26 is not particularly limited.

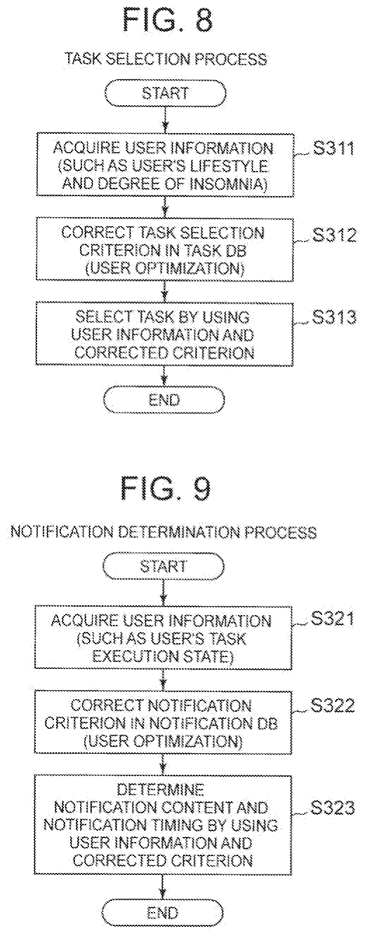

Next, the task selection process (step S202 in FIG. 7) by the task setting unit 27 will be described in more detail. FIG. 8 is a flowchart showing an example of a more detailed processing flow of the task selection process.

In the example shown in FIG. 8, first, the task DB individual adaptation unit 271 acquires user information from the personal DB 24 (step S311). Here, for example, user information including user's attributes, lifestyle, degree of insomnia, and the like inputted by the user is acquired. Note that, in a case of the task selection process for second and subsequent tasks, the acquired user information may include a sleep record of the user during previous task execution, an improvement status, and the like.

Next, the task DB individual adaptation unit 271 compares the acquired user information with user information in result data of another user stored in the result DB 26, and corrects a task selection criterion in the task DB 21. (step S312).

Hereinafter, an example of correction of the task selection criterion will be described. This example shows an example in which an effectiveness of each task is corrected as a task selection criterion. The task DB individual adaptation unit 271 first refers to the result DB 26 on the basis of the acquired user information, and searches for another user (hereinafter, a similar user) close to the target user. Here, the acquired user information is compared with user information of result data of another user stored in the result DB 26, and another user whose similarity degree is within a certain range is extracted. The similarity degree is calculated, for example, when user information of each user is converted into a feature vector on the basis of a cosine similarity degree between feature vectors, Euclidean distance, or the like.

In calculating the similarity degree, weighting may be performed for each item, for example, by increasing an influence on items related to insomnia in the questionnaire.

Next, the task DB individual adaptation unit 271 refers to the task DB 21 and optimizes a parameter of a standard effectiveness associated with each task for the target user. The following is an example of a method of optimizing the effectiveness of each task by the task DB individual adaptation unit 271 for the target user.

1. For each similar user, reference is made to a task (selected task) selected from the result DB 26, an improvement status after the end of the task, a task execution status, and the like.

2. For a selected task whose task execution status is a certain level or more, an individual effectiveness according to the improvement status after the end of the task is calculated.

3. A standard effectiveness of the task DB 21 is corrected in accordance with the individual effectiveness of the similar user. The effectiveness may be corrected, for example, by averaging the individual effectiveness of similar users. At this time, an average may be taken including the standard effectiveness of the task DB 21. In addition, the average (weighted average) may be taken after further weighting the individual effectiveness of each similar user on the basis of a similarity degree with the target user. Note that the similarity degree between with the target user can be used for a cutoff for similar users for which the average is to be taken. That is, the correction may be performed by using only the individual effectiveness of the similar user whose similarity degree is a predetermined value or more and taking an average with the standard effectiveness. Note that the correction method is merely an example, and the present invention is not limited to these methods. In this example, the corrected standard effectiveness obtained in this way is regarded as the individual effectiveness of the target user.

Finally, the task presentation unit 272 uses an effectiveness of each task after correction by the task DB individual adaptation unit 271, that is, the individual effectiveness of the target user, to select, from the tasks stored in the task DB 21, an effective task for the user information stored in the personal DB 24 (step S313). The task presentation unit 272 may, for example, present the task to the user in descending order of the effectiveness. Further, the task presentation unit 272 may be made not to present a task whose effectiveness is equal to or less than a certain value at that time.

Next, a notification determination process (step S205 in FIG. 7) by the notification unit 28 will be described in more detail. FIG. 9 is a flowchart showing an example of a more detailed processing flow of the notification determination process.

In the example shown in FIG. 9, first, the notification DB individual adaptation unit 281 acquires user information from the personal DB 24 (step S321). Here, for example, user information including user's attributes, task execution status, current improvement status, and the like inputted by the user are acquired.

Next, the notification DB individual adaptation unit 281 compares the acquired user information with user information in result data of another user stored in the result DB 26, and corrects a criterion for notification in the notification DB 22. (step S322).

Hereinafter, an example of correction of a criterion for notification will be described. This example shows an example in which correction is made on, as a criterion for notification, a determination criterion for a notification content for a task execution status or an improvement status of insomnia. First, the notification DB individual adaptation unit 281 refers to the result DB 26 on the basis of the acquired user information, and searches for a similar user. Note that the method for searching for a similar user may be similar to that in the case of correcting a task selection criterion.

Next, the notification DB individual adaptation unit 281 refers to the notification DB 22, and optimizes, for the target user, a parameter of a standard determination criterion for a notification content associated with a task currently being executed. The following is an example of a method for optimizing the determination criterion for the notification content for each task by the notification DB individual adaptation unit 281 for the target user.

1. For each similar user, reference is made to a notification content from the result DB 26, a criterion for the notification content, an improvement status before and after the notification, and the like.

2. In accordance with the improvement status of the similar user, weighting is performed on the determination criterion for the notification content of the similar user. Examples of the determination criterion for the notification content include, for example, “a criterion for encouraging task execution (for example, an execution rate less than 0%, and the like)”, “a criterion for praising for a task execution status (for example, an execution rate 0% or more, and the like)”, and “a criterion for praising for an insomnia improvement status (for example, ISI is improved by 0 points, and the like)”. The notification DB individual adaptation unit 281 may, for example, perform weighting on each of these determination criteria in accordance with the improvement status after the notification.

As an example, when the improvement status has changed to a better one after the notification, weighting is performed so that a positive evaluation is made in determining whether to select such a criterion. Whereas, when the improvement status has not changed or changed to a worse one after the notification, weighting is performed so that a negative evaluation is made in determining whether to select such a criterion. At this time, it is also possible to perform weighting on a criterion that has not been selected, in accordance with the improvement status.

Further, it is also possible to correct a criterion content itself in accordance with the improvement status in 2. described above. For example, the criterion itself may be changed in accordance with the improvement status, such as: correction is not made when the improvement status has changed to a better one after the notification; a condition (a threshold value, and the like) is lowered in the criterion to advance notification when the improvement status has not changed; and a condition is raised to reduce the notification when the improvement status has changed to a worse one. Hereinafter, a determination criterion weighted in accordance with an improvement status of a similar user is referred to as an individual determination criterion of the similar user.

3. A standard determination criterion of a notification content of the notification DB 22 is corrected in accordance with the individual determination criterion of the similar user. The standard determination criterion for a notification content may be corrected, for example, by taking a weighted average with the individual determination criterion of the similar user. At this time, the average (weighted average) may be taken after the individual determination criterion of each similar user is further weighted on the basis of a similarity degree between with the target user. Note that the similarity degree between with the target user can be used for a cutoff for similar users for which the average is to be taken. Note that the correction method is merely an example, and the present invention is not limited to these methods. In this example, the corrected standard determination criterion obtained in this way is regarded as an individual determination criterion of the target user.

Lastly, the notification execution unit 282 uses the determination criterion for the notification content corrected by the notification DB individual adaptation unit 281, that is, uses the individual determination criterion of the target user, to appropriately determine an effective notification content for the user information stored in the personal DB 24, from the notification contents stored in the notification DB 22 (step S323). Note that, when there is no notification content satisfying the determination criterion, the notification execution unit 282 may determine that there is no notification.

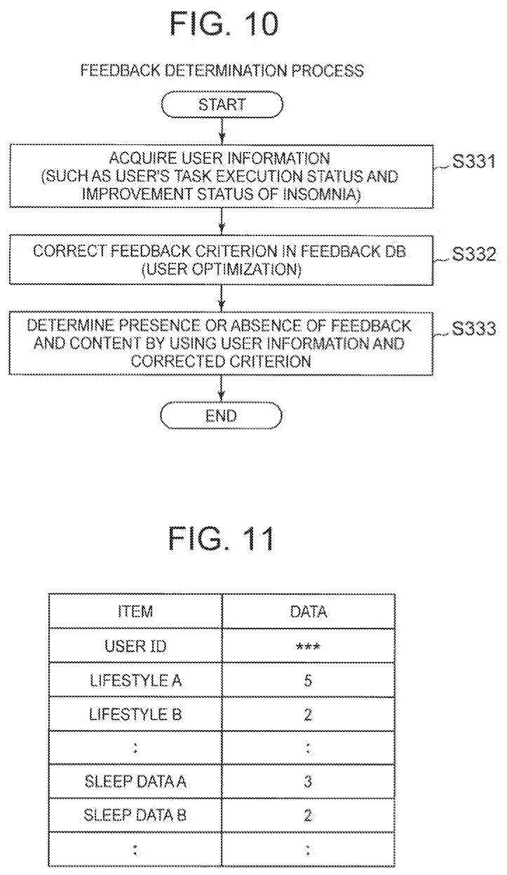

Next, a feedback determination process (step S210 in FIG. 7) by the feedback unit 29 will be described in more detail. FIG. 10 is a flowchart showing an example of a more detailed processing flow of the feedback determination process.

In the example shown in FIG. 10, first, the feedback DB individual adaptation unit 291 acquires user information from the personal DB 24 (step S331). Here, for example, user information including user's attributes, task execution status, improvement status after the task execution, and the like inputted by the user are acquired.

Next, the feedback DB individual adaptation unit 291 compares the acquired user information with user information in result data of another user stored in the result DB 26, and corrects a criterion for feedback in the feedback DB 23. (step S332).

Hereinafter, an example of correction of a criterion for feedback will be described. This example shows an example in which correction is made on, as a criterion for feedback, a determination criterion for a feedback content for an improvement status of insomnia after task execution. First, the feedback DB individual adaptation unit 291 refers to the result DB 26 on the basis of the acquired user information, and searches for a similar user. Note that the method for searching for a similar user may be similar to that in the case of correcting a task selection criterion.

Next, the feedback DB individual adaptation unit 291 refers to the feedback DB 23, and optimizes, for the target user, a parameter of a standard determination criterion for a feedback content associated with a task currently being executed. The following is an example of a method for optimizing the determination criterion for the notification content for each task by the notification DB individual adaptation unit 281 for the target user.

1. For each similar user, reference is made to a notification content from the result DB 26, a criterion for the notification content, an improvement status before and after the notification, and the like.

2. In accordance with the improvement status of the similar user, weighting is performed on the determination criterion for the feedback content of the similar user. Examples of the determination criterion for the feedback content include, for example, “a criterion for giving advice on continuation of task execution (for example, an execution rate of less than ◯%, and the like)”, “a criterion for praising for a task execution status (for example, an execution rate ◯% or more, and the like)”, “a criterion for praising for an insomnia improvement status (for example, ISI is improved by ◯ points, and the like)”, and “a criterion for setting a new task (for example, ISI is less than ◯ points, and the like)”. The feedback DB individual adaptation unit 291 may, for example, perform weighting on each of these determination criteria in accordance with the improvement status after the feedback.

A method of weighting in accordance with the improvement status after the feedback for the determination criterion for the feedback content of the similar user may be basically similar to that of the determination criterion for the notification content. Hereinafter, a determination criterion weighted in accordance with an improvement status of a similar user is referred to as an individual determination criterion of the similar user.

3. A standard determination criterion for a feedback content of the feedback DB 23 is corrected in accordance with a similarity degree between the target user and the similar user, and with the individual determination criterion of the similar user. A method for correcting the standard determination criterion for a feedback content may be basically similar to that of the standard determination criterion for a notification content. Also in this example, the corrected standard determination criterion obtained in this way is regarded as an individual determination criterion of the target user.

Lastly, the feedback execution unit 292 uses the determination criterion for the feedback content corrected by the feedback DB individual adaptation unit 291, that is, uses the individual determination criterion of the target user, to appropriately determine an effective feedback content for the user information stored in the personal DB 24, from the feedback contents stored in the feedback DB 23 (step S333). Note that, when there is no feedback content satisfying the determination criterion, the feedback execution unit 292 may determine that there is no feedback.

Next, a specific example of a task presentation process will be described with reference to FIGS. 11 to 19. FIG. 11 is an explanatory view showing an example of information stored in the personal DB 24. In the example shown in FIG. 11, in association with a user ID for identifying the user, at least an achievement degree for each prescribed item related to a lifestyle (lifestyles A and B and the like in the figure), and data for each predetermined item related to sleep (such as sleep data A and B and the like in the figure) such as average sleeping time and sleep efficiency, are stored. In this example, the achievement degree related to the lifestyle is registered in five stages. Further, data related to sleep is also registered in five stages on the basis of, for example, magnitude of numbers.

In this way, representing all items by five-stage numbers enables the use as feature vectors as they are, and makes it easy to calculate the similarity degree between users.

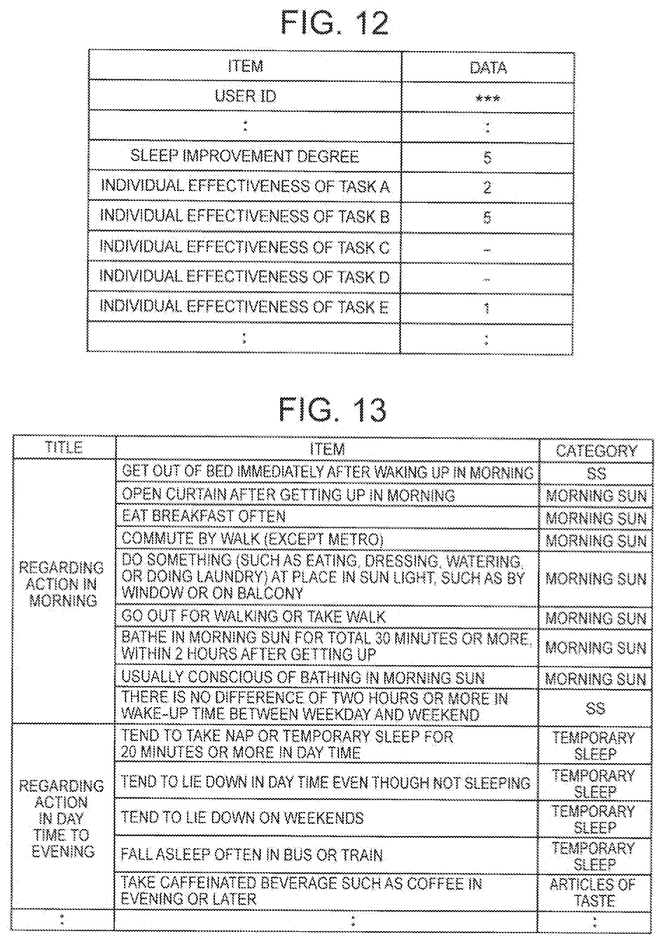

FIG. 12 is an explanatory view showing an example of information stored in the result DB 26. In the example shown in FIG. 12, at least a sleep improvement degree of the user after execution of the program and an individual effectiveness of each task are stored in association with the user ID for identifying the user. The individual effectiveness is, for example, a value calculated by, for example, multiplying a sleep improvement degree of the user after execution of the program by an execution status of the user. When the task is easy to execute and the effect is high, the individual effectiveness is set to be high. Note that unexecuted tasks are excluded from the evaluation target.

Alternatively, it is also possible to register the effectiveness of the task and the difficulty of the task separately. In that case, the effectiveness may be excluded from the evaluation target for the tasks whose execution status is a certain level or less.

Note that an example of the operation described above has shown the method of calculating the individual effectiveness of the similar user each time, but the individual effectiveness of each user may also be calculated and registered in registering the user information in the result DB 26 in this way.

FIG. 13 is an explanatory view showing an example of question items related to a user's lifestyle and sleep state. In this system, for example, the question items as shown in FIG. 13 are prepared in advance, and data related to a user's lifestyle and data on a sleep state are obtained by receiving an answer input from the user for the question items.

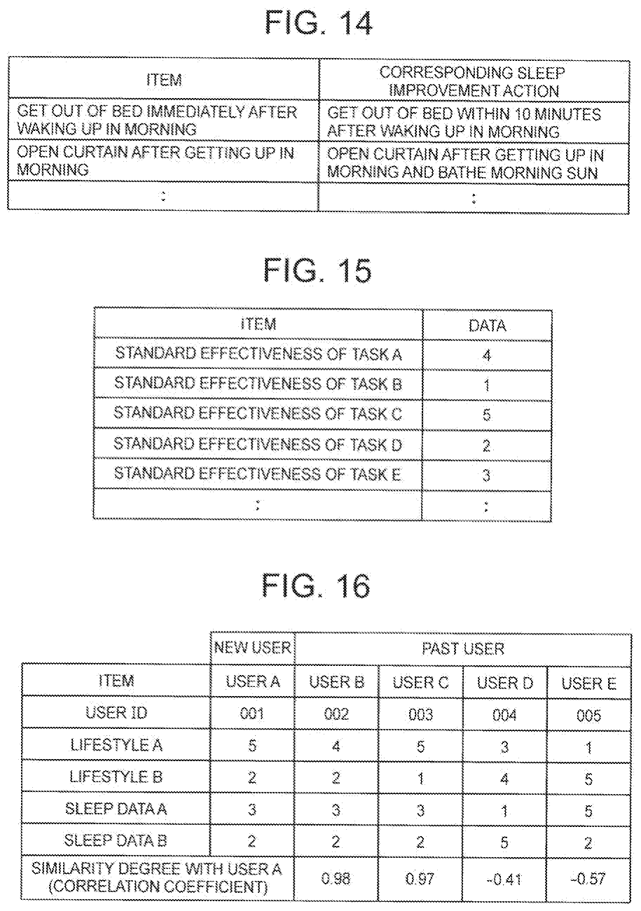

Further, FIG. 14 is an explanatory view showing an example of a sleep improvement action corresponding to each item of the question items related to a user's lifestyle and a sleep state. For example, a corresponding improvement action is prepared in advance for each item of the question items, and when the item is not fulfilled, the item may be set as a candidate for the task. Note that it is also possible to determine and register suggestion timing and the like for each item, such as an improvement action for the first week, an improvement action for the second week, and the like.

FIG. 15 is an explanatory view showing an example of information stored in the task DB 21. In the example shown in FIG. 15, a standard effectiveness of each task is stored.

FIG. 16 is an explanatory view showing an example of searching for a similar user. Now, suppose that there are a user A who is a new user, and four users (users B, C, D, and E) in the result DB. Then, it is assumed that user information of each user is as shown in FIG. 16.

In such a case, for each of the users B, C, D, and E, a correlation coefficient with the user A may be calculated, and it may be determined whether or not to be a similar user on the basis of the calculation result. In this example, it is assumed that the correlation coefficients with the user A are individually calculated as the user B: 0.98, the user C: 0.97, the user D: −0.41, and the user E: −0.57. In such a case, for example, when a threshold value for regarding as the similar user is set to 0.8, it is determined that the users B and C are similar users of the user A.

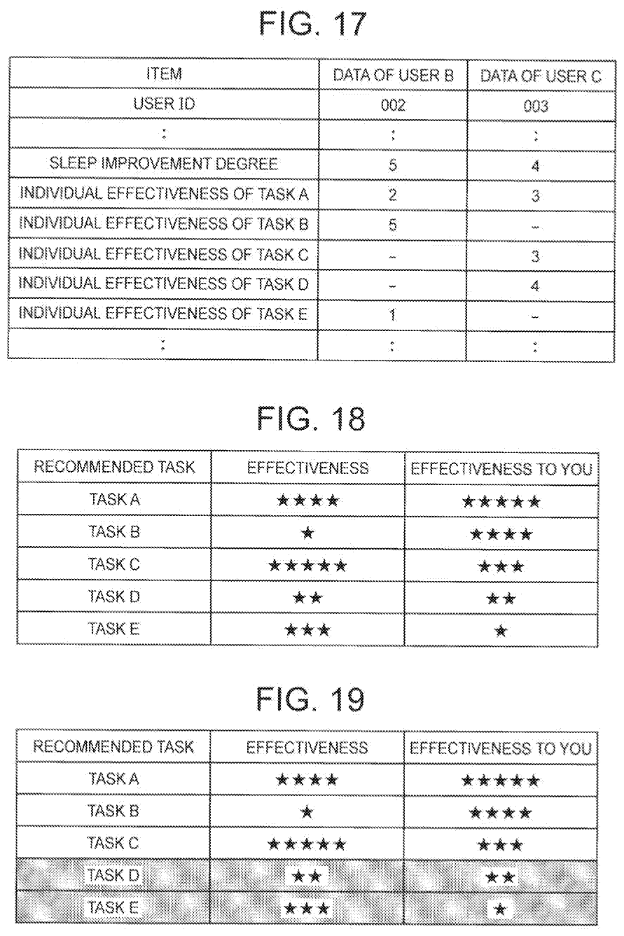

Next, description will be made on an example of calculating an individual effectiveness of a similar user, and an example of optimizing a standard effectiveness in the task DB 21 by using the individual effectiveness of the similar user. FIG. 17 is an explanatory view showing another example of information stored in the result DB 26. Now, as shown in FIG. 17, it is assumed that the individual effectiveness of the task A of the user B indicated by the result data is 2, and the individual effectiveness of the task A of the user C is 3. Note that, as shown in FIG. 15, the standard effectiveness of the task A is 4.

In such a case, consider a case of calculating the individual effectiveness of the task A for the user A. In this case, the individual effectiveness of the task A for the user A may be calculated by taking an average of the standard effectiveness and the individual effectiveness of the similar user. That is, the individual effectiveness of the task A for the user A may be calculated as, for example, (4+2+3)/3=3. Further, at this time, for example, it is also possible to assign a specific weight to the standard effectiveness, or to assign a weight according to the similarity degree with the user A to the individual effectiveness of the similar user.

In this way, the task DB individual adaptation unit 271 calculates the individual effectiveness of the target user for each task. Then, the task presentation unit 272 selects a task on the basis of the individual effectiveness of the target user for each task calculated in this way.

FIGS. 18 and 19 are explanatory views showing examples of presenting a task to the target user. For example, as shown in FIG. 18, by the task presentation unit 272 displaying the individual effectiveness of the target user as “effectiveness to you” in addition to the standard effectiveness, and presenting in an order from one having a highest individual effectiveness, it becomes easier for the user to select a task that is suitable for the user. Note that the standard effectiveness is not always necessary, but the user can use as a reference for selecting a task by comparing two types of effectiveness.

In addition, as shown in FIG. 19, when the corrected effectiveness (individual effectiveness of the target user) is a certain value or less, the task presentation unit 272 can gray out or remove the task from the options without displaying.

Note that the above has shown the example in which, in selecting a task, the standard effectiveness is corrected to the individual effectiveness of the target user on the basis of the effectiveness individually obtained on the basis of actual effects for the similar user, but the same basically applies to notification and feedback. That is, on the basis of a criterion individually obtained based on actual effects for the similar user and the effectiveness thereof, a criterion defined standardly and effectiveness thereof may simply be corrected to the individual criterion of the target user and the effectiveness thereof.

As described above, according to the present exemplary embodiment, it is possible to more appropriately and automatically perform task presentation, notification, and feedback according to a status of a user. Therefore, even in non-face-to-face, an optimal sleep improvement activity for the user can be provided to more users.

In particular, according to the present exemplary embodiment, it is possible to appropriately determine which task is appropriate (easy to execute and expected to have an effect) for each user. Therefore, for example, a more effective sleep improvement program for the user can be provided by not presenting a task with a low effect.

An optimal sleeping habit varies from user to user, and therefore some sleep improvement methods generally considered suitable may not worth executing for some users. By estimating such an effectiveness and the like based on nature and characteristics of each user on the basis of effects on similar users of the user, a more effective sleep improvement program for the user can be provided.

In addition, the sleep improvement program produces an effect when the user executes the task, but only some users can continue the task execution autonomously. Therefore, it is important to take a measure attaching importance to psychological effects on the user, such as a measure for encouraging task execution, with appropriate contents and timing. In the present exemplary embodiment, criteria are optimized for each user on the basis of effects on past users even for notification and feedback. Therefore, unlike a uniform approach, an improvement in the psychological effects is also expected.

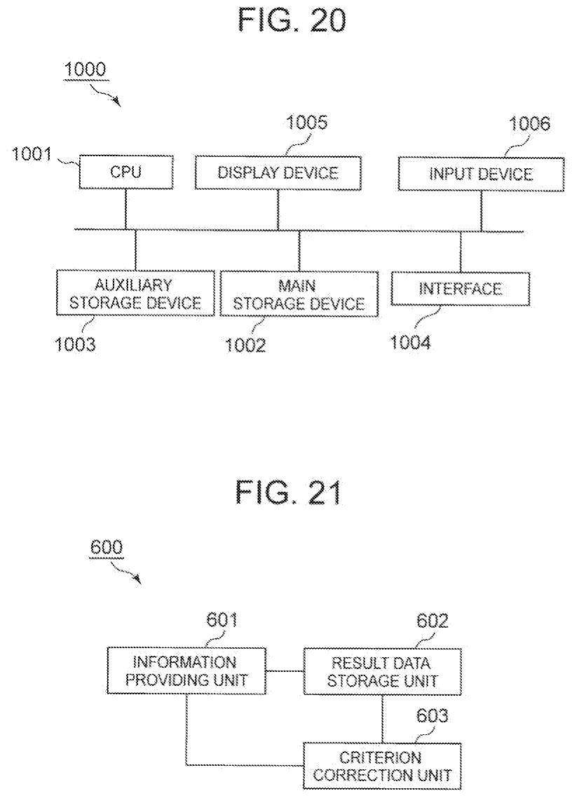

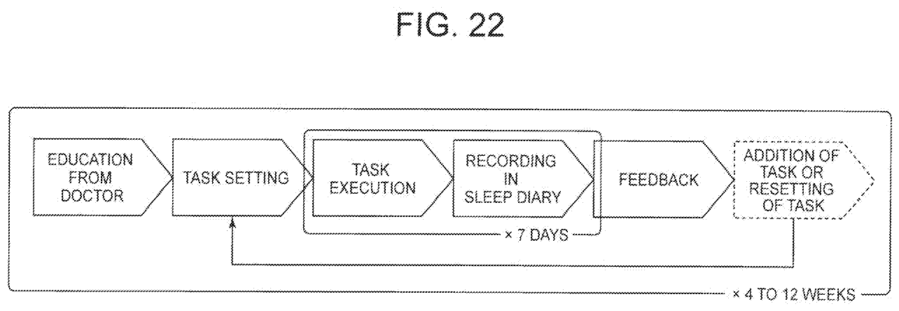

Further, FIG. 20 is a schematic block diagram showing a configuration example of a computer according to each exemplary embodiment of the present invention. A computer 1000 includes a CPU 1001, a main storage device 1002, an auxiliary storage device 1003, an interface 1004, a display device 1005, and an input device 1006.

A server and other devices and the like included in the sleep improvement assistance system of each exemplary embodiment described above may be implemented in the computer 1000. In that case, an operation of each device may be stored in the auxiliary storage device 1003 in a form of a program. The CPU 1001 reads out the program from the auxiliary storage device 1003 to develop in the main storage device 1002, and performs predetermined processing in each exemplary embodiment in accordance with the program. Note that the CPU 1001 is an example of an information processing device that operates in accordance with a program, and may include, for example, a micro processing unit (MPU), a memory control unit (MCU), a graphics processing unit (GPU), or the like, in addition to a central processing unit (CPU).
The auxiliary storage device 1003 is an example of a non-transitory tangible medium. Other examples of the non-transitory tangible medium include a magnetic disk, a magneto-optical disk, a CD-ROM, a DVD-ROM, a semiconductor memory, and the like, connected via the interface 1004. Further, when this program is distributed to the computer 1000 by a communication line, the computer 1000 that has received the distribution may develop the program in the main storage device 1002 and execute predetermined processing in each exemplary embodiment.

Further, the program may be for realizing a part of predetermined processing in each exemplary embodiment. Furthermore, the program may be a differential program that realizes predetermined processing in each exemplary embodiment in combination with another program already stored in the auxiliary storage device 1003.

The interface 1004 transmits and receives information to and from another device. Further, the display device 1005 presents information to the user. Further, the input device 1006 receives an input of information from a user.

Moreover, depending on the processing content in the exemplary embodiment, some elements of the computer 1000 can be omitted. For example, if the computer 1000 does not present information to the user, the display device 1005 can be omitted. For example, if the computer 1000 does not receive an information input from a user, the input device 1006 can be omitted.

In addition, part or all of each constituent element of each exemplary embodiment described above is implemented by a general-purpose or dedicated circuit (Circuitry), a processor, or the like, or a combination thereof. These may be configured by a single chip or may be configured by a plurality of chips connected via a bus. In addition, part or all of each constituent element of each exemplary embodiment described above may be realized by a combination of the above-described circuit and the like and a program.

When part or all of each constituent element of exemplary embodiment described above is realized by a plurality of information processing devices, circuits, and the like, the plurality of information processing devices, circuits, and the like may be arranged concentratedly or distributedly. For example, the information processing devices, the circuits, and the like may be realized as a form in which each is connected via a communication network, such as a client and server system, a cloud computing system, and the like.

Next, an outline of the present invention will be described. FIG. 21 is a block diagram showing an outline of a sleep improvement assistance system of the present invention. A sleep improvement assistance system 600 shown in FIG. 21 is particularly a sleep improvement assistance system that assists improvement of a user's sleep state through assistance with user's execution of a sleep improvement program based on CBT-I, and the sleep improvement assistance system 600 includes an information providing unit 601, a result data storage unit 602, and a criterion correction unit 603.

When user information that is information regarding sleep of a target user of a sleep improvement program based on CBT-I is inputted, the information providing unit 601 (for example, the automatic discrimination model unit 14, the task setting unit 27, the notification unit 28, the feedback unit 29) uses an automatic discrimination model that automatically determines and outputs an output suitable for the target user from a predetermined output set in accordance with a phase of a sleep improvement program of the target user, to provide information to the target user.

The result data storage unit 602 (for example, the operation data storage unit 13, the result DB 26) stores result data including at least user information and information regarding information provision performed by the information providing unit, for a past user who has finished a sleep improvement program.

The criterion correction unit 603 (for example, the individual adaptation means 141, the task DB individual adaptation unit 271, the notification DB individual adaptation unit 281, the feedback DB individual adaptation unit 291) compares user information of the target user with user information included in the result data, and corrects a criterion to be used when the automatic discrimination model determines an output suitable for a user, on the basis of a result of the comparison.

Further, the information providing unit 601 provides information to the target user by using the automatic discrimination model after the criterion is corrected by the criterion correction unit 603.

Such a configuration enables various processes that have been performed by experts in sleep improvement activities to be optimized and provided for each user

Although the present invention has been described with reference to the exemplary embodiments above, the present invention is not limited to the above-described exemplary embodiments. Various changes that can be understood by those skilled in the art can be made to the configuration and details of the present invention within the scope of the present invention.

This application claims priority based on Japanese Patent Application 2017-200933, filed on Oct. 17, 2017, the entire disclosure of which is incorporated herein.

### INDUSTRIAL APPLICABILITY

The present invention is not limited to a sleep improvement program based on CBT-I, and can be suitably applied to a program in which an optimal output varies depending on nature, characteristics, and a situation of a user.

### REFERENCE SIGNS LIST

11 User information input unit
12 Case data storage unit
13 Operation data storage unit
14 Automatic discrimination model unit
141 Individual adaptation means
15 Data output unit
21 Task DB
22 Notification DB
23 Feedback DB
24 Personal DB
25 User information input unit
26 Result DB
27 Task setting unit
271 Task DB individual adaptation unit
272 Task presentation unit
28 Notification unit
281 Notification DB individual adaptation unit
282 Notification execution unit
29 Feedback unit
291 Feedback DB individual adaptation unit
292 Feedback execution unit
1000 Computer
1001 CPU
1002 Main storage device
1003 Auxiliary storage device
1004 Interface
1005 Display device
1006 Input device
600 Sleep improvement assistance system
601 Information providing unit
602 Result data storage unit
603 Criterion correction unit

1. A sleep improvement assistance system comprising:
an information providing unit that uses an automatic discrimination model that automatically determines and outputs, when user information that is information regarding sleep of a target user of a sleep improvement program based on CBT-I is inputted, an output suitable for the target user from a predetermined output set in accordance with a phase of a sleep improvement program of the target user, to provide information to the target user;
a result data storage unit that stores result data including at least user information and information regarding information provision performed by the information providing unit, for a past user who has finished a sleep improvement program; and
a criterion correction unit that compares user information of the target user with user information included in the result data, and corrects a criterion to be used when the automatic discrimination model determines an output suitable for a user, based on a result of the comparison, wherein
the information providing unit provides information to the target user by using the automatic discrimination model after the criterion is corrected by the criterion correction unit.

2. The sleep improvement assistance system according to claim 1, wherein
the result data storage unit stores result data including information indicating an effect of a sleep improvement program, and
the criterion correction unit compares user information of the target user with user information included in the result data, and corrects the criterion, based on a difference amount or a similarity degree of the user information and based on information regarding an effect of a sleep improvement program of a past user for which the difference amount or the similarity degree is obtained.

3. The sleep improvement assistance system according to claim 2, wherein
the criterion correction unit compares user information of the target user with user information included in the result data, extracts a similar user from the result data based on the similarity degree, and corrects the criterion based on information regarding an effect of a sleep improvement program on the extracted similar user.

4. The sleep improvement assistance system according to claim 2, wherein
the criterion correction unit compares user information of the target user with user information included in the result data, extracts a similar user from the result data based on the similarity degree, and corrects the criterion based on information regarding an effect of a sleep improvement program on the extracted similar user and based on the similarity degree with the target user.

5. The sleep improvement assistance system according to claim 1, wherein
the information providing unit includes at least one of:
a task presentation unit that presents, to the target user, a task or a candidate for the task to be worked on during a sleep improvement program, by using an automatic task discrimination model that automatically determines and outputs a task suitable for the target user from a set of tasks defined in advance, based on a selection criterion that is predetermined, when user information including information regarding a lifestyle of the target user is inputted;
a notification execution unit that performs notification to the target user by using an automatic notification discrimination model that automatically determines and outputs a notification content suitable for the target user from a set of notification contents defined in advance, based on a determination criterion that is predetermined, when user information including information regarding a task execution status of the target user is inputted; or
a feedback execution unit that performs feedback to the target user by using an automatic feedback discrimination model that automatically determines and outputs a feedback content suitable for the target user from a set of feedback contents defined in advance, based on a determination criterion that is predetermined, when user information including information regarding a task execution status of the target user or an improvement status after task execution is inputted, and
based on a result of comparing user information of the target user with user information included in the result data, the criterion correction unit corrects at least one of the selection criterion used in the automatic task discrimination model, the determination criterion used in the automatic notification discrimination model, or the determination criterion used in the automatic feedback discrimination model.

6. The sleep improvement assistance system according to claim 5, wherein
the information providing unit includes the task presentation unit, and
the selection criterion used in the automatic task discrimination model includes at least a task effectiveness or a task execution difficulty.

7. The sleep improvement assistance system according to claim 5, wherein
the information providing unit includes the notification execution unit, and
the set of notification contents includes at least a notification content of praising for a task execution status or a notification content of encouraging task execution.

8. The sleep improvement assistance system according to claim 5, wherein
the information providing unit includes the feedback execution unit, and
the determination criterion used in the automatic feedback discrimination model includes at least a criterion for determining quality of a task execution status or a criterion for determining quality of an improvement status after task execution.

9. A sleep improvement assistance method comprising, by an information processing device:
using an automatic discrimination model that automatically determines and outputs, when user information that is information regarding sleep of a target user of a sleep improvement program based on CBT-I is inputted, an output suitable for the target user from a predetermined output set in accordance with a phase of a sleep improvement program of the target user, to provide information to the target user;
storing, in a predetermined result data storage unit, result data including at least user information and information regarding information provision performed by the information processing device in a sleep improvement program, for a past user who has finished a sleep improvement program; and
comparing, in using the automatic discrimination model, user information of the target user with user information included in the result data, and correcting a criterion to be used when the automatic discrimination model determines an output suitable for a user, based on a result of the comparison.

10. A non-transitory computer-readable recording medium in which a sleep improvement assistance program is recorded, the sleep improvement assistance program causing
a computer to execute:
a process of using an automatic discrimination model that automatically determines and outputs, when user information that is information regarding sleep of a target user of a sleep improvement program based on CBT-I is inputted, an output suitable for the target user from a predetermined output set in accordance with a phase of a sleep improvement program of the target user, to provide information to the target user;
a process of storing, in a predetermined result data storage unit, result data including at least user information and information regarding information provision performed by the computer in a sleep improvement program, for a past user who has finished a sleep improvement program; and
a process of comparing, in using the automatic discrimination model, user information of the target user with user information included in the result data, and correcting a criterion to be used when the automatic discrimination model determines an output suitable for a user, based on a result of the comparison.
